(19) United States
(12) Patent Application Publication
(10) Pub. No.: US 2024/0075045 A1
(43) Pub. Date: Mar. 7, 2024

(54) METHODS AND COMPOSITIONS FOR TREATING PERICARDITIS

(71) Applicant: Romeg Therapeutics LLC, Woburn, MA (US)

(72) Inventors: Indu MUNI, Woburn, MA (US); Neal MUNI, Woburn, MA (US); Naomi VISHNUPAD, Woburn, MA (US)

(21) Appl. No.: 18/257,494

(22) PCT Filed: Dec. 14, 2021
(86) PCT No.: PCT/US2021/063331

§ 371 Date: Jun. 14, 2023

Related U.S. Application Data
(60) Provisional application No. 63/125,770, filed on Dec. 15, 2020.

(51) Int. Cl. A61K 31/616; A61J 1/03; A61J 1/14; A61K 9/00; A61K 9/10; A61K 9/20; A61K 9/48; A61K 31/167; A61K 31/4439; A61P 9/00

(52) U.S. Cl. CPC A61K 31/616; A61J 1/03; A61J 1/14; A61K 9/0053; A61K 9/10; A61K 9/2013; A61K 9/2027; A61K 9/2054; A61K 9/2059; A61K 9/4825; A61K 9/4866; A61K 31/167; A61K 31/4439; A61P 9/00

(57) ABSTRACT

Compositions and methods of treating pericarditis and preventing recurrent pericarditis are provided.

### CROSS REFERENCE

This application claims the benefit of U.S. Provisional Application No. 63/125,770, filed Dec. 15, 2020, which is herein incorporated by reference in its entirety.

### BACKGROUND

The membrane enclosing the heart, referred to as the pericardium, consists of an outer fibrous layer and an inner double layer of serous membrane. The pericardium has multiple functions, including promoting cardiac efficiency by limiting acute cardiac dilation, maintaining ventricular compliance, and distributing hydrostatic forces. The pericardium also shields the heart by reducing external friction and anatomically fixes the heart through its ligamentous function. Pericarditis refers to inflammation of the pericardium. A common symptom of pericarditis is a sudden onset of sharp chest pain caused by the layers of the sac-like tissue rubbing against the heart. The pain is typically less severe when sitting up and more severe when lying down or breathing deeply. The pain may resemble that of angina but differs in that pericarditis pain changes with body position, whereas pain resulting from myocardial infarction (heart attack) is generally constant and pressure-like. Other symptoms of pericarditis may include heart palpitations, dry cough, fever, and fatigue.

The cause of pericarditis can be idiopathic (unknown) but is often due to a viral infection such as coxsackievirus, herpesvirus, mumps virus, or HIV, among others. Other causes include bacterial infections such as tuberculosis, uremic pericarditis, heart attack, cancer, autoimmune disorders, and chest trauma. Depending on the time of presentation and duration, pericarditis is divided into acute and chronic forms. Acute pericarditis can occur as a complication of infections, chest trauma, or as a result of myocardial infarction. Chronic pericarditis is usually the result of autoimmune disorders such as lupus, scleroderma and rheumatoid arthritis. A form of chronic pericarditis is constrictive pericarditis. Both types of pericarditis can disrupt the normal rhythm and function of the heart. Recurrent pericarditis is the most common and troublesome complication of acute pericarditis and occurs in 10-30% of patients with a first episode of acute pericarditis.

Treatment of acute pericarditis in most cases is with high dose non-steroidal anti-inflammatory drugs (NSAIDs), usually several times per day for up to two weeks. Steroids can be used if the current treatment is not appropriate. Additionally, colchicine has shown promise in hastening symptomatic improvement during acute pericarditis as well as in the secondary prevention of recurrent pericarditis. When used in addition to NSAIDs, colchicine may be dosed as long as a few months following an acute episode. Symptoms usually improve in a few days to weeks but can on occasion last months. Complications can include cardiac tamponade, myocarditis, and constrictive pericarditis. Therefore, there remains an unmet need for the treatment and prevention of recurrence of pericarditis.

### SUMMARY OF THE DISCLOSURE

Described herein are methods for treating or preventing pericarditis in a patient in need thereof, comprising administering to the patient a pharmaceutical composition comprising (a) colchicine and (b) aspirin. In some embodiments, the composition comprises (a) about 0.2 mg to 1.0 mg of colchicine and (b) about 500 mg to 1,000 mg of aspirin. In some embodiments, the composition comprises (a) about 0.5 mg of colchicine and (b) about 800 mg of aspirin. In some embodiments, the composition is administered as a single combined dose. In some embodiments, the composition is administered orally. In some embodiments, the composition is in the form of pill, tablet, capsule or powder. In some embodiments, the composition is an immediate release or a delayed release formulation. In some embodiments, the composition is administered QD, BID, or TID. In some embodiments, the patient is hospitalized. In some embodiments, the patient is an outpatient. In some embodiments, the composition is administered to the patient for at least 14 days. In some embodiments, the composition is administered until pericarditis associated symptoms are controlled. In some embodiments, the composition is administered to the patient as part of a regimen comprising (a) administering the composition for about 14 days; and (b) administering colchicine for an additional 3 to 26 weeks. In some embodiments, colchicine is administered at about 0.2 to about 1.0 mg per day. In some embodiments, colchicine is administered QD or BID. In some embodiments, the method further comprises administering to the patient in need thereof an additional dose of aspirin. In some embodiments, the additional dose of aspirin is from about 500 mg to about 1,000 mg. In some embodiments, the additional dose of aspirin is about 600 mg, 700 mg, 800 mg, or 900 mg. In some embodiments, the additional dose of aspirin is administered to the patient QD or BID. In some embodiments, the pericarditis is acute or recurrent. In some embodiments, the pericarditis is recurrent and the rate of recurrence is reduced following administering to the patient the pharmaceutical composition. In some embodiments, a gastroprotective agent such as a proton pump inhibitor (PPI) may be prescribed separately with a dose of aspirin. In some embodiments, the additional dose of aspirin is combined with a PPI. The PPI could be omeprazole, esomeprazole, pantoprazole or lansoprazole or their respective salts. In some embodiments, the PPI dose is 20 mg-40 mg. In some embodiments, the combination of aspirin and a PPI is administered as a combined dosage form. In some embodiments, the combination of aspirin and a PPI is enteric coated. In some embodiments, the combination of aspirin and omeprazole is enteric coated.

In another aspect provided herein is a drug delivery system comprising: a package comprising a first compartment comprising colchicine and aspirin, a second compartment comprising aspirin, and a seal separating the first compartment and the second compartment, said seal rupturing more easily under application of force than other portions of the package. In some embodiments, the aspirin is in the form of a tablet, the colchicine and aspirin is in the form of a combination tablet, or both. In some embodiments, the drug delivery system further comprises a third compartment comprising colchicine and aspirin. In some embodiments, the second compartment further comprises a proton pump inhibitor, such as a proton pump inhibitor selected from the group consisting of omeprazole, esomeprazole, lansoprazole and pantoprazole.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts two exemplary packaging presentations, an institutional use “day pack” blister card for daily treatment, and an outpatient “treatment pack” blister card for a 14-day combination treatment, followed by remaining colchicine tablets obtained from a pill bottle.

### DETAILED DESCRIPTION OF THE DISCLOSURE

Disclosed herein are methods and compositions for the treatment of pericarditis.

### Compounds and Compositions

In some aspects, disclosed herein are pharmaceutical compositions comprising (a) colchicine and (b) aspirin.

Aspirin, also known as acetylsalicylic acid (ASA), is a medication used to reduce pain, fever, and inflammation. Aspirin is a nonsteroidal anti-inflammatory drug (NSAID) and works similarly to other NSAIDs but also suppresses the normal functioning of platelets. Specific inflammatory conditions treated by aspirin include Kawasaki disease, and rheumatic fever. Aspirin given shortly after a heart attack decreases the risk of death and additionally, aspirin is also used long-term to help prevent further heart attacks, ischemic strokes, and blood clots in people at high risk.

At least two different types of cyclooxygenases, COX-1 and COX-2, are acted on by aspirin. Aspirin irreversibly inhibits COX-1 and modifies the enzymatic activity of COX-2. COX-2 normally produces prostanoids, most of which are proinflammatory. Aspirin-modified PTGS2 (Prostaglandin-endoperoxide synthase 2) produces lipoxins, most of which are anti-inflammatory.

Colchicine is a medication used to treat gout and as an anti-inflammatory agent for long-term treatment of Bechet's disease. Other uses for colchicine include the prevention of attacks of familial Mediterranean fever.

Following administration, colchicine accumulates in white blood cells and affects them in a variety of ways: decreasing motility, mobilization (especially chemotaxis) and adhesion. Generally, colchicine appears to inhibit multiple proinflammatory mechanisms, while enabling increased levels of anti-inflammatory mediators. Apart from inhibiting mitosis, colchicine inhibits neutrophil motility and activity, leading to a net anti-inflammatory effect, which has efficacy for inhibiting or preventing gout inflammation. Colchicine also interferes with the NLPR3 inflammasome complex found in neutrophils and monocytes that mediates interleukin-1 beta (IL-1β) and IL-18 activation (Slobodnick A et al., “Update on Colchicine, 2017,” Rheumatology 2018;57:i4-i11). In doing so, colchicine is presumed to have an inhibitory impact on the innate immune response that amplifies inflammatory signals and contributes to epithelial damage By virtue of its known effect on inhibiting the NLRP3 inflammasome and leukocyte activity, colchicine could have a beneficial role in disrupting the cascade of inflammatory dysregulation and hyperactivity.

Applications for colchicine's anti-inflammatory effects have been studied with regard to atherosclerosis, acute coronary syndromes, and chronic coronary disease (e.g., stable coronary artery disease/ischemic heart disease). In patients with recent myocardial infarction (recent heart attack), it has been found to reduce risk of future cardiovascular events

### Methods of Treatment

In some aspects, disclosed herein are methods for treating or preventing pericarditis in a patient in need thereof, comprising administering to the patient a pharmaceutical composition comprising (a) colchicine and (b) aspirin.

In some embodiments, the composition comprises from about 0.1 mg to about 7 mg per day or any range therein of colchicine. In some embodiments, the composition comprises from about 0.1 mg to about 5 mg per day or any range therein of colchicine. In some embodiments, the composition comprises from about 0.1 mg to about 1.2 mg or any range therein of colchicine. In some embodiments, the composition comprises from about 0.1 mg to about 1.0 mg or any range therein of colchicine. In some embodiments, the composition comprises from about 0.2 mg to about 1.0 mg or any range therein of colchicine. In some embodiments, the composition comprises about 0.1 mg, about 0.2 mg, about 0.3 mg, about 0.4 mg, about 0.5 mg, about 0.6 mg, about 0.7 mg, about 0.8 mg, about 0.9 mg, or about 1 mg of colchicine. In some embodiments, the composition comprises about 0.1 mg of colchicine. In some embodiments, the composition comprises about 0.2 mg of colchicine. In some embodiments, the composition comprises about 0.3 mg of colchicine. In some embodiments, the composition comprises about mg of colchicine. In some embodiments, the composition comprises about 0.5 mg of colchicine. In some embodiments, the composition comprises about 0.6 mg of colchicine. In some embodiments, the composition comprises about 0.7 mg of colchicine. In some embodiments, the composition comprises about 0.8 of colchicine. In some embodiments, the composition comprises about 0.9 mg of colchicine. In some embodiments, the composition comprises about 1 mg of colchicine. In some embodiments, the composition comprises about 1.2 mg of colchicine (such as, for example, two 0.6 mg colchicine doses).

In some embodiments, the composition comprises from about 100 mg to about 5,000 mg per day or any range therein of aspirin. In some embodiments, the composition comprises from about 500 mg to about 2,500 mg per day or any range therein of aspirin. In some embodiments, the composition comprises from about 500 mg to about 2,000 mg per day or any range therein of aspirin. In some embodiments, the composition comprises from about 500 mg to about 1,500 mg per day or any range therein of aspirin. In some embodiments, the composition comprises from about 500 mg to about 1,000 mg per day or any range therein of aspirin. In some embodiments, the composition comprises from about 600 mg to about 900 mg per day or any range therein of aspirin. In some embodiments, the composition comprises from about 700 mg to about 900 mg per day or any range therein of aspirin.

In some embodiments, the composition comprises about 100 mg, about 150 mg, about 200 mg, about 250 mg, about 300 mg, about 350 mg, about 400, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg, about 2,000 mg, about 2,050 mg, about 2,100 mg, about 2,150 mg, about 2,200 mg, about 2,250 mg, about 2,300 mg, about 2,350 mg, about 2,400 mg, about 2,450 mg, or about 2,500 mg of aspirin.

In some embodiments, the composition comprises about 500 mg of aspirin. In some embodiments, the composition comprises about 550 mg of aspirin. In some embodiments, the composition comprises about 600 mg of aspirin. In some embodiments, the composition comprises about 650 mg of aspirin. In some embodiments, the composition comprises about 700 mg of aspirin. In some embodiments, the composition comprises about 750 mg of aspirin. In some embodiments, the composition comprises about 800 mg of aspirin. In some embodiments, the composition comprises about 850 mg of aspirin. In some embodiments, the composition comprises about 900 mg of aspirin. In some embodiments, the composition comprises about 950 mg of aspirin. In some embodiments, the composition comprises about 1,000 mg of aspirin. In some embodiments, the composition comprises about 1,050 mg of aspirin. In some embodiments, the composition comprises about 1,100 mg of aspirin. In some embodiments, the composition comprises about 1,150 mg of aspirin. In some embodiments, the composition comprises about 1,200 mg of aspirin. In some embodiments, the composition comprises about 1,250 mg of aspirin. In some embodiments, the composition comprises about 1,300 mg of aspirin. In some embodiments, the composition comprises about 1,350 mg of aspirin. In some embodiments, the composition comprises about 1,400 mg of aspirin. In some embodiments, the composition comprises about 1,450 mg of aspirin. In some embodiments, the composition comprises about 1,500 mg of aspirin. In some embodiments, the composition comprises about 2,000 mg of aspirin.

In some embodiments, the composition comprises (a) about 0.1 mg to 3.0 mg of colchicine and (b) about 500 mg to 2,000 mg of aspirin. In some embodiments, the composition comprises (a) about 0.1 mg to 1.0 mg of colchicine and (b) about 500 mg to 1,500 mg of aspirin In some embodiments, the composition comprises (a) about 0.2 mg to 1.0 mg of colchicine and (b) about 500 mg to 1,000 mg of aspirin. In some embodiments, the composition comprises (a) about 0.2 mg to 1.0 mg of colchicine and (b) about 600 mg to 900 mg of aspirin

In some embodiments, the composition comprises (a) about 0.5 mg of colchicine and (b) about 700 mg of aspirin.

In some embodiments, the composition comprises (a) about 0.5 mg of colchicine and (b) about 800 mg of aspirin.

In some embodiments, the composition comprises (a) about 0.5 mg of colchicine and (b) about 850 mg of aspirin.

In some embodiments, the composition comprises (a) about 0.5 mg of colchicine and (b) about 900 mg of aspirin.

In some embodiments, the composition comprises (a) about 0.5 mg of colchicine and (b) about 950 mg of aspirin.

In some embodiments, the composition comprises (a) about 0.5 mg of colchicine and (b) about 1,000 mg of aspirin.

The molar ratio of (a) colchicine and (b) aspirin is not particularly restricted. For example, when the components (a) and (b) are combined in a composition, the molar ratio of them may be in the range of 1:500 to 500:1, or of 1:100 to 100:1, or of 1:50 to 50:1, or of 1:20 to 20:1, or 1:10 to 10:1, or 1:5 to 5:1, or 1:1.

In some embodiments, the combination is administered sequentially or concurrently. In some embodiments, the combination is administered concurrently. In some embodiments, the combination is administered sequentially.

In some embodiments, the combination is administered as a single combined dose. In some embodiments, the combination is administered as two separate doses.

### Dosing

In some embodiments, the composition is administered to a patient in need thereof. In some embodiments, the patient is a mammal In some embodiments, the mammal is a human.

In certain embodiments, the compositions disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some embodiments, the compositions are effective at preventing recurrence of pericarditis.

In prophylactic applications, compositions disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-1000 mg per day or from about 0.5 mg to about 500 mg per day. In one embodiment, the desired dose is conveniently presented in a single dose or in divided doses.

Typical oral dosages range from about 0.001 to about 100 mg/kg body weight per day, or any range therein. Oral dosages are usually administered in one or more dosages, typically, one to three dosages per day. The exact dosage will depend upon the frequency and mode of administration, the gender, age, weight and general health of the subject treated, the nature and severity of the condition treated and any concomitant diseases to be treated and other factors evident to those skilled in the art.

In some embodiments, the composition comprising colchicine and aspirin is administered from about 0.1 mg/kg to about 500 mg/kg per day. In some embodiments, the composition is administered at about 0.1 mg/kg, about 0.3 mg/kg, about 0.5 mg/kg, about 0.8 mg/kg, about 1 mg/kg, about 5 mg/kg, about 10 mg/kg, about 20 mg/kg, about 50 mg/kg, about 100 mg/kg, about 150 mg/kg, about 200 mg/kg, about 300 mg/kg, about 400 mg/kg, or about 500 mg/kg per body weight of the patient.

In some embodiments, the composition comprising colchicine and aspirin is administered at about 0.1 mg/kg per body weight. In some embodiments, the composition comprising colchicine and aspirin is administered at about 0.3 mg/kg per body weight. In some embodiments, the composition comprising colchicine and aspirin is administered at about 0.5 mg/kg per body weight. In some embodiments, the composition comprising colchicine and aspirin is administered at about 1 mg/kg per body weight. In some embodiments, the composition comprising colchicine and aspirin is administered at about 5 mg/kg per body weight. In some embodiments, the composition comprising colchicine and aspirin is administered at about 10 mg/kg per body weight. In some embodiments, the composition comprising colchicine and aspirin is administered at about 25 mg/kg per body weight. In some embodiments, the composition comprising colchicine and aspirin is administered at about 50 mg/kg per body weight. In some embodiments, the composition comprising colchicine and aspirin is administered at about 75 mg/kg per body weight. In some embodiments, the composition comprising colchicine and aspirin is administered at about 100 mg/kg per body weight. In some embodiments, the composition comprising colchicine and aspirin is administered at about 125 mg/kg per body weight. In some embodiments, the composition comprising colchicine and aspirin is administered at about 150 mg/kg per body weight. In some embodiments, the composition comprising colchicine and aspirin is administered at about 200 mg/kg per body weight. In some embodiments, the composition comprising colchicine and aspirin is administered at about 300 mg/kg per body weight. In some embodiments, the composition comprising colchicine and aspirin is administered at about 400 mg/kg per body weight. In some embodiments, the composition comprising colchicine and aspirin is administered at about 500 mg/kg per body weight.

In some embodiments, the pharmaceutical compositions are presented in a unit dosage form by methods known to those skilled in the art.

In some embodiments, the composition is administered parenterally. In some embodiments, the composition is administered as an intravenous infusion or as an injection. In some embodiments, the composition is administered by intravenous infusion.

In some embodiments, the composition is administered orally.

In some embodiments, the composition is administered in the form of pill, tablet, or capsule. In some embodiments, the composition is administered in the form of a combination tablet comprising colchicine and aspirin. In some embodiments, an additional dose of aspirin is prescribed as 800 mg aspirin or a combination of 800 mg aspirin and 20 mg omeprazole. In some embodiments, an additional dose of aspirin is prescribed as 800 mg aspirin or a combination of 800 mg aspirin and 40 mg omeprazole.

In some embodiments, the composition is administered once per day (QD), twice per day (BID) or three times per day (TID), four times a day (QID), or more. In some embodiments, the composition is administered QD, BID, or TID. In some embodiments, the composition is administered QD or BID. In some embodiments, the composition is administered QD. In some embodiments, the composition is administered BID. In some embodiments, the composition is administered TID.

In some embodiments, the composition is administered to the patient for between about to about 30 days, or any range therein. In some embodiments, the composition is administered to the patient for between about 14 to about 30 days. In some embodiments, the composition is administered to the patient for between about 14 to about 21 days.

In some embodiments, the composition is administered to the patient for about 7 days, about 10 days, about 14 days or about 21 days. In some embodiments, the composition is administered to the patient for about 7 days. In some embodiments, the composition is administered to the patient for about 10 days. In some embodiments, the composition is administered to the patient for about 11 days. In some embodiments, the composition is administered to the patient for about 12 days. In some embodiments, the composition is administered to the patient for about 13 days. In some embodiments, the composition is administered to the patient for about 14 days. In some embodiments, the composition is administered to the patient for about 15 days. In some embodiments, the composition is administered to the patient for about 16 days. In some embodiments, the composition is administered to the patient for about 17 days. In some embodiments, the composition is administered to the patient for about 18 days. In some embodiments, the composition is administered to the patient for about 19 days. In some embodiments, the composition is administered to the patient for about 20 days. In some embodiments, the composition is administered to the patient for about 21 days. In some embodiments, the composition is administered to the patient for about 1 month.

In some embodiments, the composition is administered to the patient for at least 7 days, at least 10 days, at least 14 days or at least 21 days. In some embodiments, the composition is administered to the patient for at least 10 days. In some embodiments, the composition is administered to the patient for at least 14 days. In some embodiments, the composition is administered to the patient for at least 15 days. In some embodiments, the composition is administered to the patient for at least 16 days. In some embodiments, the composition is administered to the patient for at least 17 days. In some embodiments, the composition is administered to the patient for at least 18 days. In some embodiments, the composition is administered to the patient for at least 19 days. In some embodiments, the composition is administered to the patient for at least 20 days. In some embodiments, the composition is administered to the patient for at least 21 days.

In some embodiments, the patient is hospitalized. In some embodiments, the composition is administered to the patient for the duration of a hospital stay.

In some embodiments, the patient is receiving medical treatment without being admitted to a hospital (including outpatients). In some embodiments, the patient is an out-patient.

In some embodiments, the composition is administered until symptoms associated with pericarditis are controlled. In some embodiments, the composition is administered until at least one pericarditis-associated symptom is controlled. Pericarditis-associated symptom include, but are not limited to, chest pain, fever, weakness, fatigue, coughing, difficulty breathing, pain when swallowing, and heart palpitations.

In some embodiments, the composition is administered to the patient in need thereof as part of a regimen comprising (a) administering the compositions described herein for about 14 days; and (b) further administering colchicine for an additional 3 to 26 weeks after the administration of the composition.

In some embodiments, colchicine is further administered after the administration of the composition for about 3 to about 26 weeks, or any range therein. In some embodiments, colchicine is administered after the administration of the composition for about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, or about 26 weeks. In some embodiments, colchicine is administered after the administration of the composition for about 3 weeks. In some embodiments, colchicine is administered after the administration of the composition for about 4 weeks. In some embodiments, colchicine is administered after the administration of the composition for about 8 weeks. In some embodiments, colchicine is administered after the administration of the composition for about 12 weeks. In some embodiments, colchicine is administered after the administration of the composition for about 16 weeks. In some embodiments, colchicine is administered after the administration of the composition for about 20 weeks. In some embodiments, colchicine is administered after the administration of the composition for about 24 weeks. In some embodiments, colchicine is administered after the administration of the composition for about 26 weeks.

In some embodiments, colchicine is further administered after the administration of the further administered at about 0.2 mg, about 0.3 mg, about 0.4 mg, about 0.5 mg, about 0.6, about about 0.8, about 0.9, or about 1.0 mg per day. In some embodiments, colchicine is further administered after the administration of the further administered at about 0.2 mg per day. In some embodiments, colchicine is further administered after the administration of the further administered at about 0.3 mg per day. In some embodiments, colchicine is further administered after the administration of the further administered at about 0.4 mg per day. In some embodiments, colchicine is further administered after the administration of the further administered at about 0.5 mg per day. In some embodiments, colchicine is further administered after the administration of the further administered at about 0.6 mg per day. In some embodiments, colchicine is further administered after the administration of the further administered at about 0.7 mg per day. In some embodiments, colchicine is further administered after the administration of the further administered at about 0.8 mg per day. In some embodiments, colchicine is further administered after the administration of the further administered at about 0.9 mg per day.

In some embodiments, colchicine is further administered after the administration of the further administered at about 0.2 mg, about 0.3 mg, about 0.4 mg, about 0.5 mg, about 0.6, about about 0.8, about 0.9, or about 1.0 mg per dose. In some embodiments, colchicine is further administered after the administration of the further administered at about 0.2 mg per dose. In some embodiments, colchicine is further administered after the administration of the further administered at about 0.3 mg per dose. In some embodiments, colchicine is further administered after the administration of the further administered at about 0.4 mg per dose. In some embodiments, colchicine is further administered after the administration of the further administered at about 0.5 mg per dose. In some embodiments, colchicine is further administered after the administration of the further administered at about 0.6 mg per dose.

In some embodiments, colchicine is further administered after the administration of the composition QD or BID. In some embodiments, colchicine is further administered QD. In some embodiments, colchicine is further administered BID.

In some embodiments, a patient is administered three tablets per day, with one tablet comprising aspirin and the other two tablets comprising aspirin and colchicine. In some embodiments, a patient is administered three tablets per day, with one tablet comprising aspirin and a proton pump inhibitor (such as omeprazole) and the other two tablets comprising aspirin and colchicine. In some embodiments, the aspirin tablet, the aspirin and proton pump inhibitor tablet, and the aspirin and colchicine tablet appear similar. In some embodiments, the aspirin tablet, the aspirin and proton pump inhibitor tablet, and the aspirin and colchicine tablet appear identical.

In some embodiments, the regimen further comprises optionally administering to the patient in need an additional dose of aspirin during step (a) of the regimen. In some embodiments, aspirin is optionally co-administered with the compositions described herein for about 14 days.

In some embodiments, the regimen further comprises optionally administering from about 500 mg to about 1,000 mg of aspirin. In some embodiments, the regimen further comprises optionally administering about 600 mg, about 700 mg, about 800 mg, about 800 mg, about 900 mg of aspirin. In some embodiments, the regimen further comprises optionally administering about 600 mg of aspirin. In some embodiments, the regimen further comprises optionally administering about 700 mg of aspirin. In some embodiments, the regimen further comprises optionally administering about 800 mg of aspirin. In some embodiments, the regimen further comprises optionally administering about 800 mg of aspirin. In some embodiments, the regimen further comprises optionally administering about 900 mg of aspirin.

In some embodiments, the regimen further comprises optionally administering an additional dose of aspirin QD or BID. In some embodiments, the regimen further comprises optionally administering an additional dose of aspirin QD. In some embodiments, the regimen further comprises optionally administering an additional dose of aspirin BID.

In some embodiments, the regimen further comprises optionally administering an additional dose of aspirin concurrently or sequentially with the composition described herein.

### Pharmaceutical Compositions

The present disclosure also comprises salts of both (a) colchicine and (b) aspirin, typically pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in Berge, S. M. et al., J Pharm. Sci. 1977, 66, 2, the contents of which are hereby incorporated by reference.

The composition of the disclosure may be administered alone or in combination with pharmaceutically acceptable carriers, diluents or excipients, in either single or multiple doses. The pharmaceutical compositions according to the disclosure may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, PA, 2013.

In some embodiments, the pharmaceutical compositions may be formulated with preservatives, adjuvants and carriers. Preservatives include anti-microbials, anti-oxidants, and agents that enhance sterility. Exemplary preservatives include ascorbic acid, ascorbyl palmitate, benzyl alcohol, BHA, BHT, citric acid, erythorbic acid, fumaric acid, malic acid, propyl gallate, sodium ascorbate, sodium benzoate, sodium bisulfate, sodium metabisulfite, sodium sulfite, parabens (methyl-, ethyl-, butyl-), benzoic acid, potassium sorbate, and vanillin.

In some embodiments, buffering systems and additional preservatives may be used. The solution is formulated to inhibit growth of bacteria, mold, and yeast for storage at room temperature and ambient conditions. In some embodiments, surfactants are used. In some embodiments, surfactants such as poloxamer 407 are used. In some embodiments, the solutions and suspensions described herein comprise a surfactant or wetting agent selected from the group consisting of alcohol, glycerin, propylene glycol, polyethylene glycol, mineral oil, benzalkonium chloride, benzethonium chloride, cetylpyridinium chloride, docusate sodium, nonoxynol 9, octoxynol, poloxamer, poloxamer 124, poloxamer 188, poloxamer 237, poloxamer 338, poloxamer 407, polyoxyl 35 castor oil, polyoxyl 40 hydrogenated castor oil, polyoxyl 10 oleyl ether, polyoxyl 20 cetylstearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol, and combinations thereof. In some embodiments, the compositions includes anti-foaming agents such as simethicone and simethicone emulsion. In some embodiments, the compositions described herein comprise a humectant selected from the group consisting of glycerin, hexylene glycol, propylene glycol, and sorbitol. In some embodiments, glycerin can be used to produce humectant properties.

In some embodiments, the pharmaceutical compositions includes buffering agents. Non-limiting examples of buffering agents include, but are not limited to, sodium bicarbonate, potassium bicarbonate, magnesium hydroxide, magnesium lactate, magnesium gluconate, aluminum hydroxide, aluminum hydroxide/sodium bicarbonate precipitate, a mixture of an amino acid and a buffer, a mixture of aluminum glycinate and a buffer, a mixture of acid salt and an amino acid and a buffer, and a mixture of an alkali salt of an amino acid and a buffer. Additional buffering agents include citric acid, sodium citrate, sodium tartarate, sodium acetate, sodium carbonate, sodium polyphosphate, potassium polyphosphate, sodium pyrophosphate, potassium pyrophosphate, disodium hydrogen phosphate, dibasic sodium phosphate, trisodium phosphate, tripotassium phosphate, sodium acetate, potassium metaphosphate, magnesium oxide, magnesium carbonate, magnesium silicate, calcium acetate, calcium glycerophosphate, calcium chloride, calcium hydroxide, calcium lactate, calcium carbonate, calcium bicarbonate, and other calcium salts.

The pharmaceutical compositions may be specifically formulated for administration by any suitable route, such as oral, rectal, nasal, pulmonary, topical (including buccal and sublingual), transdermal, intracisternal, intraperitoneal, vaginal, and parenteral (including subcutaneous, intramuscular, intrathecal, intravenous, and intradermal) routes. It will be appreciated that the route will depend on the general health and age of the subject to be treated, the nature of the condition to be treated, and the active ingredient. In some embodiments, the pharmaceutical composition is formulated for oral administration to a subject. In some embodiments, the pharmaceutical composition is formulated as a tablet or pill. In some embodiments, the pharmaceutical composition is formulated as a solid tablet suitable for oral administration to a subject. In some embodiments, the pharmaceutical composition is formulated as a solid combination tablet comprising colchicine and aspirin.

Pharmaceutical compositions for oral administration include solid dosage forms such as capsules, tablets, dragees, pills, lozenges, powders, and granules. Where appropriate, the compositions may be prepared with coatings, such as enteric coatings or they may be formulated so as to provide controlled release of the active ingredient such as sustained or prolonged release according to methods well known in the art. Liquid dosage forms for oral administration include solutions, emulsions, suspensions, syrups, and elixirs, either manufactured as such, or as a solid form for reconstitution prior to use.

Pharmaceutical compositions for parenteral administration include sterile aqueous and non-aqueous injectable solutions, dispersions, suspensions, or emulsions as well as sterile powders to be reconstituted in sterile injectable solutions or dispersions prior to use. Other suitable administration forms include, but are not limited to, suppositories, sprays, ointments, creams, gels, inhalants, dermal patches, and implants.

The present disclosure also provides a process for making a pharmaceutical composition comprising admixing a therapeutically effective amount of a compound of the present disclosure and at least one pharmaceutically acceptable carrier or diluent.

For parenteral administration, solutions of the compositions of the present disclosure are dissolved in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intramuscular, subcutaneous and intraperitoneal administration. The compositions of the present disclosure may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatine, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions of the present disclosure are then readily administered in a variety of dosage forms suitable for the disclosed routes of administration. The formulations may conveniently be presented in unit dosage form by methods known in the art of pharmacy.

Pharmaceutical compositions of the present disclosure suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatine capsule in powder or pellet form or it may be in the form of a troche or lozenge. The solid carrier may be coated or uncoated. In some embodiments, the preparation is in the form of a coated tablet. In some embodiments, the preparation is in the form of an enteric coated tablet.

If a liquid carrier is used, the preparation may be in the form of a syrup, emulsion, soft gelatin capsule or sterile injectable liquid such as an aqueous or non-aqueous liquid suspension or solution.

The pharmaceutical compositions of the disclosure may be prepared by conventional methods in the art. For example, tablets may be prepared by mixing the active ingredient with ordinary adjuvants and/or diluents and subsequently compressing the mixture in a conventional tableting machine prepare tablets. Examples of adjuvants or diluents comprise: corn starch, potato starch, talcum, magnesium stearate, gelatin, lactose, gums, and the like. Any other adjuvants or additives usually used for such purposes such as colorings, flavorings, preservatives etc. may be used provided that they are compatible with the active ingredients.

In some embodiments, the composition is an immediate release or a delayed release formulation. A delayed release formulation can be formulated to delay a compound's action for an extended period of time. A delayed release form can be formulated to delay the release of an effective dose of one or more compounds, for example, for about 4, about 8, about 12, about 16, or about 24 hours. In some embodiments, the composition is a controlled release formulation. A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

The pharmaceutical composition comprises at least 10%, 20%, 30%, 40%, 50%, 60%, 70%, 80%, 90%, 95%, or 100% by weight of a combination of (a) colchicine and (b) aspirin. The pharmaceutical composition comprises at least about 1% to about 90% by weight of a combination of (a) colchicine and (b) aspirin.

In some embodiments, the pharmaceutical compositions disclosed herein are stored at controlled room temperature (20-25° C.). In some embodiments, the pharmaceutical composition disclosed herein are protected from light.

### Combination Therapies

The treatment of certain diseases or conditions (e.g., pericarditis) in a patient or subject with pharmaceutical compositions described herein encompass additional therapies and treatment agents. Such additional therapies and treatment regimens include another therapy, e.g., NSAIDS, steroids, antibiotics, or antivirals for the treatment of the particular disease. In some embodiments, the additional therapeutic is a non-steroidal anti-inflammatory drug (NSAID). NSAIDs are members of a drug class that reduce pain, decrease fever, prevent blood clots, and decrease inflammation. Common NSAIDs include but are not limited to ibuprofen, naproxen, diclofenac, celecoxib, mefenamic acid, etoricoxib, and indomethacin. In some embodiments, the additional therapeutic is a steroid (e.g. prednisone).

In some embodiments, the additional therapeutic is a gastroprotective agent such as a proton pump inhibitor (PPI). The PPI may be prescribed separately with a dose of aspirin. In some embodiments, the additional dose of aspirin is combined with a PPI. The PPI could be omeprazole, esomeprazole, pantoprazole or lansoprazole or their respective salts. The PPI dose is 20 mg-40 mg. In some embodiments, the combination of aspirin and a PPI is enteric coated. In some embodiments, the combination of aspirin and omeprazole is enteric coated. In some embodiments, a patient is treated with a combination tablet comprising about 0.5 mg colchicine and about 800 mg aspirin, and a combination tablet comprising about 800 mg aspirin and between 20-40 mg of a PPI. In some embodiments, the PPI is omeprazole.

The term “administered simultaneously”, as used herein, is not specifically restricted and means that the compositions of the present disclosure and the additional active agent are substantially administered at the same time, e.g. as a mixture or in immediate subsequent sequence.

The term “administered sequentially”, as used herein, is not specifically restricted and means that the compositions of the present disclosure and the additional active agent are not administered at the same time but one after the other, or in groups, with a specific time interval between administrations. The time interval may be the same or different between the respective administrations of the compositions of the present disclosure and the additional active agent and may be selected, for example, from the range of 2 minutes to 96 hours, 1 to 7 days or one, two, or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

In some embodiments, the additional therapy is cardiac surgery. In some embodiments, the compositions described herein are administered for treating acute coronary syndrome, myocardial infarction, stroke, and/or coronary stent implantation, and are administered in combination with additional antiplatelet or anticoagulants (such as clopidogrel, heparin, glycoprotein IIb/IIIa inhibitors, among others).

### Drug Delivery Systems

In another aspect, described herein is a drug delivery system comprising: a package having a first compartment comprising colchicine, a second compartment comprising aspirin, and a seal separating the first compartment and the second compartment, said seal rupturing more easily under application of force than other portions of the package.

In another aspect, described herein is a drug delivery system comprising: a package having a first compartment comprising one or more combined doses of colchicine and aspirin, a second compartment comprising aspirin, and a seal separating the first compartment and the second compartment, said seal rupturing more easily under application of force than other portions of the package. In some embodiments, the package having the first compartment comprises one, two, or three individual doses of a composition of colchicine and aspirin as described herein. In some embodiments, the package having the first compartment comprises one, two, or three individual doses of a composition of colchicine and aspirin or a combined dose of aspirin and omeprazole as described herein. In some embodiments, the first compartment comprises two individual doses of the composition described herein. In some embodiments, the first compartment comprises three individual doses of the compositions described herein.

In some embodiments, the package comprises at least 7 first compartments separated with a seal, wherein said seal rupturing more easily under application of force than other portions of the package.

In some embodiments, the aspirin is in the form of a tablet, the colchicine is in the form of a tablet, or both. In some embodiments, the aspirin and the colchicine are in the form of a combined tablet, pill, or capsule. In some embodiments, disclosed herein are blister packs comprising seven days of doses of the compositions described herein, wherein each dose comprises one tablet comprising aspirin and two tablets comprising aspirin and colchicine. In some embodiments, disclosed herein are blister packs comprising seven days of doses of the compositions described herein, wherein each dose comprises one tablet comprising aspirin and a proton pump inhibitor and two tablets comprising aspirin and colchicine.

### Definitions

Unless defined otherwise, all technical and scientific terms used herein have the same meanings as commonly understood by one of ordinary skill in the art. Although any methods and materials similar or equivalent to those described herein can be used in the practice of testing of embodiments described herein, certain preferred methods, devices, and materials are now described.

As used herein and in the appended claims, the singular forms “a”, “an”, and “the” include plural reference unless the context clearly dictates otherwise. Thus, for example, reference to “an excipient” is a reference to one or more excipients and equivalents thereof known to those skilled in the art, and so forth.

The term “about” is used to indicate that a value includes the standard level of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or”. The terms “comprise”, “have”, and “include” are open-ended linking verbs. Any forms or tenses of one or more of these verbs “comprises,” “comprising,” “has,” “having,” “includes,” and “including” are also open-ended. For example, any method that “comprises,” “has” or “includes” one or more steps is not limited to possessing only those one or more steps and also covers other unlisted steps.

“Optional” or “optionally” may be taken to mean that the subsequently described structure, event or circumstance may or may not occur, and that the description includes instances where the events occurs and instances where it does not.

As used herein, the term “therapeutic” means an agent utilized to treat, combat, ameliorate, prevent or improve an unwanted condition or disease of a patient.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with an oral composition as described herein. “Administering” a composition may be accomplished by injection, topical administration, and oral administration or by other methods alone or in combination with other known techniques.

As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof In a preferred embodiment, the patient is a primate. In certain embodiments, the primate or subject is a human. In certain instances, the human is an adult. In certain instances, the human is child. In certain instances, the human is elderly. In other instances, the human is 65 years of age or older. Other examples of subjects include experimental animals such as mice, rats, dogs, cats, goats, sheep, pigs, and cows.

By “pharmaceutically acceptable”, it is meant the carrier, diluent or excipient must be compatible with the other ingredients of the formulation and not deleterious to the recipient thereof

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; prevention of recurrence of the disorder or disease; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

The following examples are provided to better illustrate the claimed methods and compositions and are not to be interpreted as limiting the scope of the methods and compositions described herein. To the extent that specific materials are mentioned, it is merely for purposes of illustration and is not intended to limit the methods and compositions described herein. One skilled in the art may develop equivalent means or reactants without the exercise of inventive capacity and without departing from the scope of the methods and compositions described herein.

### Example A-1: Parenteral Pharmaceutical Composition

To prepare a parenteral pharmaceutical composition suitable for administration by injection (subcutaneous or intramuscular), 1-1000 mg of a water-soluble salts of both aspirin and colchicine, or a pharmaceutically acceptable salt or solvate thereof, are dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Suspension

To prepare a pharmaceutical composition for oral delivery, a sufficient amount of both aspirin and colchicine, or pharmaceutically acceptable salts thereof, are added to water (with optional solubilizer(s),optional buffer(s) and taste masking excipients) to provide a 20 mg/mL solution.

### Example A-3: Oral Tablet

A tablet is prepared by mixing 20-50% by weight of a pharmaceutical composition described herein, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100 -500 mg.

### Example A-4: Oral Capsule

To prepare a pharmaceutical composition for oral delivery, 1-1000 mg of a pharmaceutical composition described herein, is mixed with starch or other suitable powder blend. The mixture is incorporated into an oral dosage unit such as a hard gelatin capsule, which is suitable for oral administration.

In another embodiment, 1-1000 mg of a pharmaceutical composition described herein, is placed into a Size 4 capsule, or a Size 1 capsule (hypromellose or hard gelatin) and the capsule is closed.

### Example 2: Treatment of Pericarditis in Humans

### Summary

This study is a multi-center, randomized, double-blind, active and placebo-controlled clinical trial of the safety and efficacy of treatments of acute pericarditis and prevention of recurrence. The study is a multi-center trial that will be conducted in 50 sites. Adult (>18 year-old) patients with acute pericarditis will be randomized between 3 treatment arms, each to be given aspirin versus colchicine versus a composition comprising a combination of colchicine and aspirin.

The primary objective of the study is to evaluate the clinical efficacy and safety of the composition of colchicine and aspirin versus each monotherapy arm (colchicine only and aspirin only, respectively) in patients with pericarditis, to assess benefits in the reduction of acute symptom persistence, as well as, the prevention of pericarditis recurrence.

Inclusion criteria:

Adult male or female subjects at least 18 years of age and not more than 80 years of age.

At least 2 of the following 4 criteria observed for the diagnosis of acute pericarditis: 1) chest pain; 2) pericardial rub; 3) electrocardiogram (ECG) changes; and 4) new or worsening pericardial effusion (2015 European Society of Cardiology Guidelines for pericardial diseases). Subject presenting with:

idiopathic acute pericarditis, or

acute pericarditis resulting from post pericardiotomy syndrome after coronary artery bypass surgery or valve surgery

Subjects with scores of greater than 2 on the 7-point Patient Global Impression of Pericarditis Severity (PGI-PS) scale for pericardial symptoms.

Subjects with acute pericarditis who are already receiving treatment with a non-steroidal anti-inflammatory drug (NSAID) alone.

Subjects with a history of intermittent, recurrent pericarditis who recovered and presenting with a new episode of pericarditis and are already receiving treatment with an NSAID alone.

Female subjects must be:

Postmenopausal, defined as at least 12 months after the cessation of menses (without an alternative medical cause) OR

Permanently sterile following documented hysterectomy, bilateral salpingectomy, bilateral oophorectomy, or bilateral tubal ligation OR

If of childbearing potential, must agree to use a highly effective method of contraception during the study and for 3 months after the last study drug administration (i.e., hormonal contraceptives associated with inhibition of ovulation, intrauterine device, intrauterine hormone-releasing system).

Subjects must be able to provide an informed consent

Exclusion criteria:

Has a history of another episode of acute pericarditis within the past 6 weeks.

Subjects with acute pericarditis who are already receiving treatment with steroids, colchicine, or biologic immunomodulators.

Pregnant or nursing women.

Life expectancy of less than 12 months.

Current treatment with systemic corticosteroids or underlying diseases requiring treatment with systemic corticosteroids.

Concomitant usage of antiplatelet and anticoagulant medication including heparin during 14-day therapy with aspirin.

Contraindication or hypersensitivity to colchicine, aspirin, or NSAIDs.

Has a history of organ transplantation, immunosuppression, including positive human immunodeficiency virus (HIV) test results.

Liver disease or transaminase levels>1.5 times the upper limit of the normal range.

Blood dyscrasias.

Has a history of autoimmune diseases (e.g., lupus or rheumatoid arthritis).

Subjects with a systemic fungal infection.

Subjects with a known pericardial effusion of tuberculous, purulent, or neoplastic causes.

Has a history of malignancy of any organ system within the past 5 years before screening (other than a successfully treated non-metastatic cutaneous squamous cell carcinoma or basal cell carcinoma and/or localized carcinoma in situ of the cervix).

Has received immunization with a live (attenuated) vaccine within 12 weeks before screening or is expected to receive live (attenuated) vaccine during the study or within 12 weeks after the last study drug administration.

Meets the following TB criteria:

History of active TB prior to screening OR

History of latent TB that was not adequately treated prior to screening OR

Signs or symptoms suggestive of active TB (e.g., new cough of >14 days in duration or change in chronic cough, persistent fever, unintentional weight loss, or night sweats) upon review of medical history and/or physical examination at screening OR

Recent close contact with a person with active TB.

Has a known or suspected current active infection or a history of chronic or recurrent infectious disease, including, but not limited to: COVID-19, chronic renal infection, chronic chest infection, sinusitis, recurrent urinary tract infection, or an open, draining infected skin wound.

Has ulcerative colitis, Crohn's disease, peptic ulcer disease or other chronic gastrointestinal disease.

Has myopericarditis, as indicated by an elevation in the serum troponin level.

Has acute coronary syndrome, as indicated by an elevation in the serum troponin level with electrocardiogram changes suggesting myocardial ischemia.

In the investigator's opinion, has a history of alcoholism or drug/chemical abuse within 2 years before screening.

In the investigator's opinion, has any other medical condition that could adversely affect the subject's participation or interfere with study evaluations. This includes significant concomitant illnesses such as, but not limited to, cardiac, renal, neurological, endocrinological, metabolic, pulmonary, gastrointestinal, or psychiatric diseases.

Has a history of myeloproliferative disorder.

Has a history of demyelinating disease or symptoms suggestive of multiple sclerosis.

Subjects with chronic kidney disease requiring hemodialysis (End-stage renal failure).

Anemia with hemoglobin concentrations of <8 g/dL.

### Experimental:

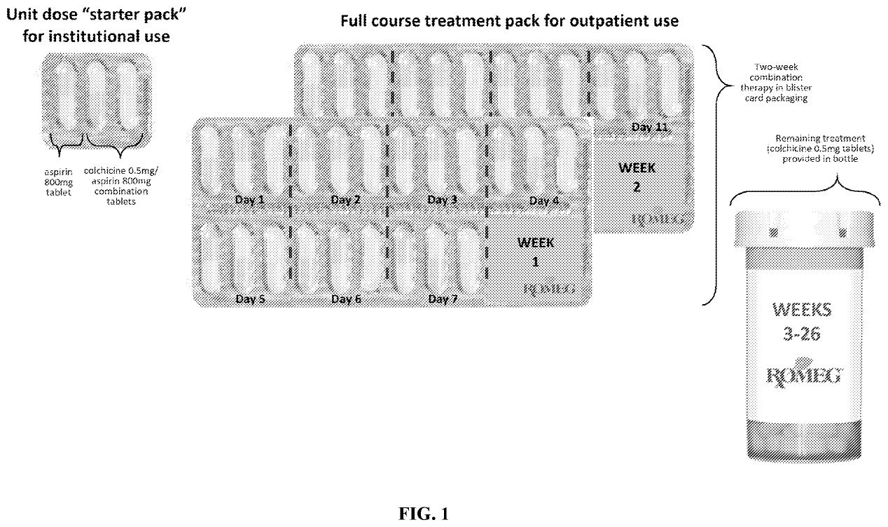

An oral dose of (a) 0.5 mg of colchicine and (b) 800 mg of aspirin will be administered for 14 days total followed by 26 weeks of 0.5 mg colchicine. As shown in FIG. 1, the 0.5 mg colchicine/800 mg aspirin dose is in the form of a combination tablet which reduces pill burden (going from 5 tablets from 2 different prescriptions down to 3 tablet), provides compliance-enhancing packaging, and is compact and easy to store. This provides a standardized treatment in a user-friendly packaging to facilitate patient adherence.

Primary Outcomes: Three co-primary endpoints are to be considered:

The first co-primary endpoint is the time to recurrence which is compared between the aspirin arm and the combination therapy arm of aspirin and colchicine. Onset of a recurrent event must be more than 30 days after randomization and is documented by recurrent pain and one or more of the following signs: fever, pericardial friction rub, electrocardiographic changes, echocardiographic evidence of pericardial effusion, and elevations in ESR or CRP. An independent Clinical Endpoint Committee (CEC) will adjudicate all recurrent pericardial events.

The second co-primary endpoint, which will be compared between the combination therapy arm and the aspirin treatment arm, is symptom persistence of pericarditis at 72 hours after treatment initiation. Symptom persistence is defined as a score of 2 or greater at the first post-baseline assessment as measured on the 7-point PGI-PS scale. All subjects treated with combination therapy or aspirin only will be eligible for an assessment of pericarditis recurrence irrespective of any symptom persistence at 72 hours.

The third co-primary endpoint is symptom persistence of pericarditis at 72 hours after treatment initiation, adjusted for the use of analgesics. This endpoint will be compared between the combination therapy arm and the colchicine arm.

Secondary Outcomes: There endpoint include:
Hospital length of stay during the 14-day period following treatment initiation
Rate of re-hospitalization
   Supportive endpoints will include physician and subject assessments/surveys:
Reduction from Screening/Baseline in pericardial pain (11-point Numeric Rating Scale [NRS])
Change from Screening/Baseline in EuroQol-5 Dimension (EQ-5D) Health Status Measurement
Change from Screening/Baseline in 36-Item Short Form (SF-36) Survey of Patient Health
Change from Screening/Baseline in Insomnia Severity Index (ISI)—American Thoracic Society
Change from Screening/Baseline in PGI-PS
Absence or reduction from Screening/Baseline of pericardial activity based on Physician Global Assessment of Pericarditis Activity (PGA-PA)
Patient satisfaction with packaging and ease-of-use

### Example 3. Manufacture of Colchicine and Aspirin Combination Tablet

A combination tablet comprising colchicine and aspirin can be produced with the following exemplary composition according to Table 1.

TABLE 1
Colchicine/Aspirin (0.5 mg/800 mg) Composition.

| | | Amount | |
|---|---|---|---|
| Ingredients | Function | mg/Tablet | % w/w |
| Colchicine, USP^{a} | Active | 0.5 | 0.04 |
| Aspirin/Starch/Povidone DC-90^{b} | Active | 876.23 | 66.97 |
| (Acetylsalicylic acid granulated) | | | |
| Microcrystalline Cellulose NF | Binder/Filler | 151.5 | 11.58 |
| (Avicel PH-102) | | | |
| Lactose Monohydrate NF | Binder/Filler | 90.11 | 6.89 |
| (Lactose 316 Fast Flo) | | | |
| Pregelatinized Starch NF | Disintegrant | 12.0 | 0.92 |
| (Starch 1500) | | | |
| Povidone K-30 NF | Binder | 8.38^{c} | 0.31^{c} |
| (Kollidon 30) | | | |
| Sodium Starch Glycolate NF | Disintegrant | 24.0 | 1.83 |
| Magnesium Stearate NF | Lubricant | 10.0 | 0.76 |
| Copovidone NF | Binder | 60.0 | 4.59 |
| (Kollidon VA 64) | | | |
| Corn Starch NF | Binder/ | 109.85^{d} | 2.90 |
| (Unipure F) | Disintegrant | | |
| Colloidal Silicon Dioxide NF | Glidant | 6.0 | 0.46 |
| (Aerosil 200) | | | |
| Stearic Acid NF | Lubricant | 10.0 | 0.76 |
| Opadry II Blue 85F90614 | Colorant | 26.0 | 1.99 |
| Purified Water USP^{e} | Vehicle | 134.0 | Not |
| | | | applicable |
| Total Tablet Weight | — | 1308.34 | 100.0 |

^{a}Quantity to be adjusted with Avicel PH-102 for 100% Assay.
^{b}Adjusted to 100% potency; based on 91.3% aspirin in aspirin/starch/povidone DC-90.
^{c}Aspirin/starch/povidone DC-90 contains 0.5% povidone (4.38 mg/tablet).
^{d}Aspirin/starch/povidone DC-90 contains 8.2% corn starch (71.85 mg/tablet).
^{e}Evaporated during processing; not part of finished product.

A flow chart of the manufacturing process for the colchicine/aspirin combination tablet (0.5 mg/800 mg) is described in Table 2.

TABLE 2
Flow Chart of Manufacturing Process for Colchicine/Aspirin Tablets,
0.5 mg/800 mg.
Dispensing of Final Blend for Aspirin Tablets, 800 mg and Final Blend
for Colchicine Tablets, 0.5 mg

[symbol]

Load half quantity of Final Blend for Aspirin Tablets, 800 mg in V
blender. Then load required quantity of Final Blend for Colchicine
Tablets, 0.5 mg. After, load remaining half Final Blend for Aspirin
Tablets, 800 mg in the V blender.

[symbol]

Blend for 3 minute at 25 RPM ± 1 RPM

[symbol]

Unload the Final Blend into fiber drums double-lined with polyethylene
bags

[symbol]

Set the tablet press with the appropriate tooling

[symbol]

Load the Usable Final Blend into the hopper of the tablet press and
Compress tablets of desired weight

[symbol]

Color Coating: Suspension: Mix Opadry Color and Water using arrow
mixer for NLT 45 minutes.

[symbol]

Load the compressed tablets into the coating pan and spray color coating
suspension onto the tablets to achieve desired weight gain.

[symbol]

Unload the coated tablets into container double lined with polyethylene
bags

### Example 4. Manufacture of Colchicine Tablet

A colchicine tablet can be produced with the following exemplary composition according to Table 3.

TABLE 3
Colchicine Tablets, 0.5 mg Composition.

| | | Amount | |
|---|---|---|---|
| Ingredients | Function | (mg/Tablet) | % w/w |
| Colchicine USP^{a} | Active | 0.5 | 0.49 |
| Microcrystalline Cellulose NF | Binder/Filler | 27.5 | 26.96 |
| (Avicel PH-102) | | | |
| Lactose Monohydrate NF | Binder/Filler | 50.0 | 49.02 |
| (Lactose 316 Fast Flo) | | | |
| Pregelatinized Starch NF | Disintegrant | 12.0 | 11.77 |
| (Starch 1500) | | | |
| Povidone K-30 NF | Binder | 4.0 | 3.92 |
| (Kollidon 30) | | | |
| Sodium Starch Glycolate NF | Disintegrant | 4.0 | 3.92 |
| Magnesium Stearate NF | Lubricant | 2.0 | 1.96 |
| Opadry II Yellow 85F120077 | Colorant | 2.0 | 1.96 |
| Purified Water USP^{b} | Vehicle | 38.0 | Not applicable |
| Total Tablet Weight | — | 102.0 | 100.0 |

^{a}Quantity to be adjusted with Avicel PH-102 for 100% Assay.
^{b}Evaporated during processing; not part of finished product

A flow chart of the manufacturing process for a colchicine tablet is described in Table 4.

TABLE 4
Flow Chart of Manufacturing Process for Colchicine Tablets, 0.5 mg.
Dispensing of Raw Materials

[symbol]

Screen Lactose Monohydrate Part 1 (54%) and Part 2 (46%) and
Pregelatinized Starch separately through # 30 mesh.

[symbol]

Granulating Solution: Dissolve Povidone K-30 in Purified water using an
arrow mixer.

[symbol]

Mix Colchicine with Part 1 (10%) of Avicel PH-102 and pass it through #
30 mesh (Active Pre-mix)

[symbol]

Pass the Part 2 (90%) of Avicel PH-102 through # 30 mesh and then pass
the pre-mix from above step through # 30 mesh (Active Mix)

[symbol]

Load screened Lactose Monohydrate Part 1 (54%) in Hobart Mixer and
prime for 1 minute at slow speed 1

[symbol]

Load above screened Active mix in Hobart Mixer and mix for 5 minute at
slow speed 1

[symbol]

Load screened Lactose Monohydrate Part 2 (46%) and Pregelatinized
Starch 1500 in Hobart Mixer and mix for 5 minute at slow speed 1

[symbol]

Granulate the powder in Hobart mixer using the granulating solution.
Add solution in 3 minutes including rinse at Hobart mixer speed 1. Add
additional water if required to get desired granulation consistency

[symbol]

Dry the wet mass in tray dryer at 50° C. till LOD is below 2%
(at 105° C.)

[symbol]

Pass dried granules through 25# mesh screen and note down weight. Pass
not passing granules through multi mill using 1.5 mm screen @1500 rpm
and screen through 25# mesh

[symbol]

Screen Sodium Starch glycolate through # 30 mesh

[symbol]

Load the milled granules from Step 10 and screened Sodium Starch
glycolate in the V-blender and blend for 5 minute at 25 RPM ± 1 RPM

[symbol]

Screen Magnesium Stearate through # 40 mesh and mix with some
material from V blender,

[symbol]

Load in the V-Blender and blend for 3 minute at 25 RPM ± 1 RPM

[symbol]

Unload the Final Blend into fiber drums double-lined with polyethylene
bags

[symbol]

Set the tablet press with the appropriate tooling

[symbol]

Load the Usable Final Blend into the hopper of the tablet press and
Compress tablets of desired weight

[symbol]

Color Coating Suspension: Mix Opadry Color and Water using arrow
mixer for NLT 45 minutes

[symbol]

Load the compressed tablets into the coating pan and spray color coating
suspension onto the tablets to achieve desired weight gain

[symbol]

Unload the coated tablets into container double lined with polyethylene
bags

### Example 5. Manufacture of Aspirin Tablet

An aspirin tablet can be produced with the following exemplary composition according to Table 5.

TABLE 5
Aspirin Tablets, 800 mg Composition.

| | | Amount | |
|---|---|---|---|
| Ingredients | Function | mg/Tablet | % w/w |
| Aspirin/Starch/Povidone DC-90^{a} | Active | 876.23 | 66.72 |
| (Acetylsalicylic acid granulated) | | | |
| Microcrystalline Cellulose NF | Binder/Filler | 189.0 | 14.39 |
| (Avicel PH-102) | | | |
| Lactose Monohydrate NF | Binder/Filler | 50.11 | 3.82 |
| (Lactose 316 Fast Flo) | | | |
| Sodium Starch Glycolate NF | Disintegrant | 20.0 | 1.52 |
| Magnesium Stearate NF | Lubricant | 8.0 | 0.61 |
| Povidone K-30 NF | Binder | 4.38^{b} | Not |
| | | | applicable^{b} |
| Copovidone NF | Binder | 80.0 | 6.09 |
| (Kollidon VA 64) | | | |
| Corn Starch USP | Binder/ | 119.85^{c} | 3.65^{e} |
| (Unipure F) | Disintegrant | | |
| Colloidal Silicon Dioxide NF | Glidant | 6.0 | 0.46 |
| (Aerosil 200) | | | |
| Stearic Acid NF | Lubricant | 10.0 | 0.76 |
| Opadry II White 85F18422 | Colorant | 26.0 | 1.98 |
| Purified Water USPd | Vehicle | 104.0 | Not |
| | | | applicable |
| Total Tablet Weight | — | 1313.34 | 100.00 |

^{a}Adjusted to 100% potency; based on 91.3% aspirin in aspirin/starch/povidone DC-90.
^{b}Aspirin/starch/povidone DC-90 contains 0.5% povidone (4.38 mg/tablet).
^{c}Aspirin/starch/povidone DC-90 contains 8.2% corn starch (71.85 mg/tablet).
^{d}Evaporated during processing; not part of the final product.

A flow chart of the manufacturing process for an aspirin tablet is described in Table 6.

A flow chart of the manufacturing process for a final blend of the aspirin tablet is described in Table 7.

A placebo/aspirin tablet can be produced with the following exemplary composition according to Table 8.

TABLE 8
Placebo/Aspirin Tablets, 800 mg Composition.

| | | Amount | |
|---|---|---|---|
| Ingredients | Function | mg/Tablet | % w/w |
| Aspirin/Starch/Povidone DC-90^{a} | Active | 876.23 | 66.97 |
| (Acetylsalicylic acid granulated) | | | |
| Microcrystalline Cellulose NF | Binder/Filler | 152.0 | 11.62 |
| (Avicel PH-102) | | | |
| Lactose Monohy drate NF | Binder/Filler | 90.11 | 6.89 |
| (Lactose 316 Fast Flo) | | | |
| Pregelatinized Starch NF | Disintegrant | 12.0 | 0.92 |
| (Starch 1500) | | | |
| Povidone K-30 NF | Binder | 8.38^{b} | 0.31 |
| (Kollidon 30) | | | |
| Sodium Starch Glycolate NF | Disintegrant | 24.0 | 1.83 |
| Magnesium Stearate NF | Lubricant | 10.0 | 0.76 |
| Copovidone NF | Binder | 60.0 | 4.59 |
| (Kollidon VA 64) | | | |
| Corn Starch NF | Binder/ | 109.85^{c} | 2.90 |
| (Unipure F) | Disintegrant | | |
| Colloidal Silicon Dioxide NF | Glidant | 6.0 | 0.46 |
| (Aerosil 200) | | | |
| Stearic Acid NF | Lubricant | 10.0 | 0.76 |
| Opadry II Blue 85F90614 | Colorant | 26.0 | 1.99 |
| Purified Water USP^{d} | Vehicle | 134.0 | Not |
| | | | applicable |
| Total Tablet Weight | — | 1308.34 | 100.00 |

^{a}Adjusted to 100% potency; based on 91.3% aspirin in aspirin/starch/povidone DC-90.
^{b}Aspirin/starch/povidone DC-90 contains 0.5% povidone (4.38 mg/tablet).
^{c}Aspirin/starch/povidone DC-90 contains 8.2% corn starch (71.85 mg/tablet).
^{d}Evaporated during processing; not part of finished product.

A flow chart of the manufacturing process for an placebo/aspirin tablet is described in Table 9.

TABLE 9
Flow Chart of Manufacturing Process for Placebo/Aspirin Tablets, 800 mg.
Dispensing of Final Blend for Aspirin Tablets, 800 mg and Final Blend
for Placebo for Colchicine Tablets

[symbol]

Load half quantity of Final Blend for Aspirin Tablets, 800 mg in V
blender. Then load required quantity of Final Blend for Placebo for
Colchicine Tablets. After it, load remaining half Final Blend for Aspirin
Tablets, 800 mg in the V blender.

[symbol]

Blend for 3 minutes at 25 RPM ± 1 RPM

[symbol]

Unload the Final Blend into fiber drums double-lined with polyethylene
bags

[symbol]

Set the tablet press with the appropriate tooling

[symbol]

Load the Usable Final Blend into the hopper of the tablet press and
Compress tablets of desired weight

[symbol]

Color Coating: Suspension: Mix Opadry Color and Water using arrow
mixer for NLT 45 minutes.

[symbol]

Load the compressed tablets into the coating pan and spray color coating
suspension onto the tablets to achieve desired weight gain.

[symbol]

Unload the coated tablets into container double lined with polyethylene
bags

A flow chart of the manufacturing process for the final blend of a placebo/aspirin tablet is described in Table 10.

TABLE 10
Flow Chart of Manufacturing Process for Final Blend of Placebo Tablets.
Dispensing of Raw Materials

[symbol]

Screen Lactose Monohydrate, Avicel PH-102, Povidone K-30 and
Pregelatinized Starch separately through # 30 mesh.

[symbol]

Granulating Solution: Dissolve Povidone K-30 in Purified water using an
arrow mixer.

[symbol]

Load screened Lactose Monohydrate in Hobart Mixer and prime for 1
minute at slow speed 1.

[symbol]

Load screened Avicel PH-102 and Pregelatinized Starch in Hobart Mixer
and mix for 10 minutes at slow speed 1

[symbol]

Granulate the powder in Hobart mixer using the granulating solution.
Add solution in 3 minutes including rinse at Hobart mixer speed 1. Add
additional water if required to get desired granulation consistency

[symbol]

Dry the wet mass in tray dryer at 50° C. till LOD is below 2%
(at 105° C.)

[symbol]

Pass dried granules through # 25 mesh screen and note down weight. Pass
not passing granules through multi mill using 1.5 mm screen @1500 rpm
and screen through # 25 mesh

[symbol]

Screen Sodium Starch glycolate through # 30 mesh

[symbol]

Load the milled granules and screened Sodium Starch glycolate in the
V-blender and blend for 5 minutes at 25 RPM ± 1 RPM

[symbol]

Screen Magnesium Stearate through # 40 mesh and mix with some
material from V blender,

[symbol]

Load in the V-Blender and blend for 3 minutes at 25 RPM ± 1 RPM

[symbol]

Unload the Final Blend into fiber drums double-lined with polyethylene
bags

[symbol]

Set the tablet press with the appropriate tooling

[symbol]

Load the Usable Final Blend into the hopper of the tablet press and
Compress tablets of desired weight

[symbol]

Color Coating: Suspension: Mix Opadry Color and Water using arrow
mixer for NLT 45 minutes.

[symbol]

Load the compressed tablets into the coating pan and spray color coating
suspension onto the tablets to achieve desired weight gain.

[symbol]

Unload the coated tablets into container double lined with polyethylene
bags

While preferred embodiments of the present methods and compositions described herein have been shown, it will be obvious to those skilled in the art that such embodiments are provided by way of example only. Numerous variations, changes, and substitutions will now occur to those skilled in the art without departing from the methods and compositions described herein. It should be understood that various alternatives to the embodiments of the methods and compositions described herein may be employed in practicing the methods and compositions described herein. It is intended that the following claims define the scope of the methods and compositions described herein and that methods and structures within the scope of these claims and their equivalents be covered thereby.

What is claimed is:

1. A method for treating or preventing pericarditis in a patient in need thereof, comprising administering to the patient a pharmaceutical composition comprising (a) colchicine and (b) aspirin.

2. The method of claim 1, wherein the composition comprises (a) about 0.2 mg to 1.0 mg of colchicine and (b) about 500 mg to 1,000 mg of aspirin.

3. The method of claim 1 or 2, wherein the composition comprises (a) about 0.5 mg of colchicine and (b) about 800 mg of aspirin.

4. The method of any one of claims 1-3, wherein the composition is administered as a single combined dose.

5. The method of any one of claims 1-3, wherein the composition is administered sequentially or simultaneously.

6. The method of any one of claims 1-5, wherein the composition is administered orally.

7. The method of any one of claims 1-6, wherein the composition is in the form of pill, tablet, capsule or powder.

8. The method of any one of claims 1-7, wherein the composition is an immediate release or a delayed release formulation.

9. The method of any one of claims 1-8, wherein the composition is administered QD, BID, or TID.

10. The method of any one of claims 1-9, wherein the patient is hospitalized.

11. The method of any of claims 1-9, wherein the patient is an outpatient.

12. The method of any one of claims 1-11, wherein the composition is administered to the patient for at least 14 days.

13. The method of any one of claims 1-11, wherein the composition is administered until pericarditis associated symptoms are controlled.

14. The method of any one of claims 1-11, wherein the composition is administered to the patient as part of a regimen comprising (a) administering the composition for about 14 days; and (b) further administering colchicine for an additional 3 to 26 weeks.

15. The method of claim 14, wherein colchicine is administered at about 0.2 to about 1.0 mg per day.

16. The method of claim 14 or 15, wherein colchicine is administered QD or BID.

17. The method of any one of claims 1-16, the method optionally further comprising administering to the patient in need thereof an additional dose of aspirin.

18. The method of claim 17, wherein the additional dose of aspirin is from about 500 mg to about 1,000 mg.

19. The method of claim 17, wherein the additional dose of aspirin is about 600 mg, 700 mg, 800 mg, or 900 mg.

20. The method of any one of claims 17-19, wherein the additional dose of aspirin is administered to the patient QD or BID.

21. The method of claim 1, wherein the pericarditis is acute or recurrent.

22. The method of claim 21, wherein the pericarditis is recurrent and a rate of recurrence is reduced following administering to the patient the pharmaceutical composition.

23. The method of any one of claims 1-3, further comprising a gastroprotective agent.

24. The method of claim 23, wherein the gastroprotective agent is a proton pump inhibitor.

25. The method of claim 24, wherein the proton pump inhibitor is selected from the group consisting of omeprazole, esomeprazole, lansoprazole and pantoprazole.

26. The method of any of claims 17-19, wherein the additional dose of aspirin further comprises a proton pump inhibitor.

27. The method of claim 26, wherein the additional dose of aspirin further comprising a proton pump inhibitor is administered as a combined dosage form.

28. A drug delivery system comprising: a package comprising a first compartment comprising colchicine and aspirin, a second compartment comprising aspirin, and a seal separating the first compartment and the second compartment, said seal rupturing more easily under application of force than other portions of the package.

29. The drug delivery system of claim 28, wherein the aspirin is in the form of a tablet, the colchicine is in the form of a tablet, or both.

30. The drug delivery system of claim 28 or 29, further comprising a third compartment comprising colchicine and aspirin.

31. The drug delivery system of any of claims 28-30, wherein the second compartment further comprises a proton pump inhibitor.

32. The drug delivery system of claim 31, wherein the proton pump inhibitor is selected from the group consisting of omeprazole, esomeprazole, lansoprazole and pantoprazole.
